(19) United States
(12) Patent Application Publication
(10) Pub. No.: US 2023/0172724 A1
(43) Pub. Date: Jun. 8, 2023

(54) EXPANDABLE INTERBODY IMPLANT DEVICE

(71) Applicant: Incite Innovations, LLC, Dallas, TX (US)

(72) Inventor: Theodore Eric Hansen, Dallas, TX (US)

(73) Assignee: Incite Innovations, LLC, Dallas, TX (US)

(21) Appl. No.: 17/542,378

(22) Filed: Dec. 4, 2021

(51) Int. Cl. A61F 2/44

(52) U.S. Cl. CPC A61F 2/447; A61F 2002/30579

(57) ABSTRACT

Disclosed herein are aspects of expandable interbody devices having an unobstructed graft material containment space. Such expandable interbody devices have a structure in which an overall plan view area of the graft material containment space is devoid of elements of the expandable interbody device enabling adjustment of the expandable interbody devices between a collapsed configuration and displaced configuration (e.g., an expanded configuration and/or a tilted configuration). In this manner, expandable interbody devices configured in accordance with embodiments of the disclosures made herein do not have obstructions within the graft material containment space thereof that limit the available volume of graft material and associated bony material growth that may be contained within the graft material containment space.

### FIELD OF THE DISCLOSURE

The disclosures made herein relate generally to interbody implant devices adapted for facilitating repair of ailments to bony structures in a living body and, more particularly, to expandable spinal interbody implant devices.

### BACKGROUND

The spinal column of humans and other species of the subphylum vertebrata is a complex structure comprised of multiple soft connective tissues and bony connective tissues well known to be subject to structural ailment. Involved in numerous functions, the spinal column provides structural support to the body, allows for functional movement of the body, and protection of delicate parts of the neurological system including the spinal cord and nerve roots. Body weight and biomechanical forces are transferred through the spinal column during most functional activities of life. Numerous complex structures constituting the spinal column permit for transfer of forces to the body in a fashion that allows motion in all three geometric planes (sagittal, coronal, axial) while simultaneously achieving the aforementioned functions.

The spinal column includes a plurality of bony vertebrae, cartilaginous intervertebral discs, and ligaments that provide for interconnection of and spacing between the segments of the spinal column. Each intervertebral disc includes a cartilaginous anulus fibrosus and nucleus pulposus. All vertebral endplates (i.e., the endplates) are each affixed to one of twenty three intervertebral discs from the inferior endplate of the second cervical vertebrae to the superior endplate of the first sacral vertebrae. Normal intervertebral discs exhibit sufficient properties (e.g., mass, density, resiliency, etc.) for providing spatial positioning and a cushioning effect between adjacent vertebrae. Thus, in a healthy spinal column, spatial positioning of and cushioning between adjacent vertebrae are maintained through such interconnection of the vertebrae by the intervertebral discs. In the lumbar region, the shape of healthy intervertebral discs are important to the overall sagittal alignment and balance of the spinal column.

It is well-known that functionality of one or more of the intervertebral discs can fully or partially fail. Causes of intervertebral disc failure may include but are not limited to degenerative, traumatic, infectious, oncologic, or congenital etiologies. Failure of intervertebral discs may result in loss of spinal column structural integrity. A loss of normal spinal column spatial geometry such as intervertebral height, intervertebral slippage, or intervertebral malalignment may result as a consequence of intervertebral disc failure. Intervertebral disc failure may also result in the loss of mechanical cushioning between vertebral segments. The consequences of intervertebral disc failure may lead to a person developing pain, numbness, weakness, or loss of neurologic functions.

Spinal implants intended to address ailments arising from failure of intervertebral disc functionality are well known in the art. Expandable interbody implant devices (sometimes referred to as expandable cages) are one particular type of such spinal implants. Expandable interbody implant devices are configured to replace or augment an intervertebral disc and provide for at least its associated intersegmental/intervertebral positioning functionality. An expandable interbody implant device is retained in fixed relationship to the adjacent vertebrae by mechanical and/or biological means. Interbody implant devices are also often configured to have or enable restoration of appropriate intervertebral endplate angles, i.e. the lordotic/kyphotic angle. The expandability of such implants allows placement thereof into a corresponding surgically-enhanced disc space between two adjacent vertebrae and, thereafter, be selectively expanded to achieve restoration or enhancement of intervertebral spacing and intervertebral angle.

When utilizing an expandable interbody device, it is generally highly desirable to provide for biological interconnection between the adjacent vertebrae for the purpose of promoting fusion of the adjacent vertebrae (i.e., such a device sometimes referred to as an expandable interbody fusion device). To this end, it is desirable to introduce a graft material between spaced-apart endplate surfaces of the adjacent vertebrae and it is desirable for the expandable interbody device to include a graft passage extending therethrough within which a portion of the graft material can reside and through which connective bony tissue can ultimately grow and join the vertebral bone directly above the expandable interbody device to the vertebral bone directly below the expandable interbody device. Any physical element of the expandable interbody device residing within the graft passage are obstructions that limit the available volume of graft material that may be contained therein and that correspondingly limits the amount of bone tissue that may ultimately grow therein. Any physical element of the expandable interbody device residing within the graft passage are obstructions that limit the direct pathway thru which bone tissue can grow across. Thus, such obstructions within the graft window are counterproductive and undesirable to the objective of vertebral interbody fusion.

Therefore, an implantable device configured for use in repairing an ailment in a bony structure of a living body (e.g., an expandable interbody implant device configured for use in repairing an ailment in a person's spinal column) that does not have elements thereof residing within the graft passage thereof is advantageous, desirable and useful.

### SUMMARY OF THE DISCLOSURE

Embodiments of the disclosures made herein are directed to expandable interbody implant devices having an unobstructed graft passage. Specifically, an expandable interbody implant device configured in accordance with embodiments of the disclosures made herein has a structure in which an entire space of the graft passage is devoid of elements thereof that enable adjustment of the expandable interbody implant device between a collapsed configuration and displaced configurations (e.g., an expanded configuration and/or a tilted configuration). In this manner, expandable interbody implant devices configured in accordance with embodiments of the disclosures made herein do not have obstructions within the graft passage thereof that limit the available volume of graft material and associated bone tissue or other soft tissue that may grow within the graft passage.

In one or more embodiments, an implant device comprises endplates and endplate movement mechanisms. The endplates are engaged with each other for enabling movement of the endplates to a collapsed configuration and to displaced configurations. The endplates jointly define a graft passage when the endplates are in the collapsed configuration. The graft passage is at least partially encompassed by a plurality of graft passage sidewalls each extending from a perimeter edge portion of a graft material containment space of a respective one of the endplates. The endplate movement mechanisms are each located entirely external to the graft passage and operably coupled to each of the endplates to enable adjustment of the endplates to the collapsed configuration and to the displaced configurations.

In one or more embodiments, an implant device comprises first and second endplates and spaced apart endplate movement mechanisms. The first and second endplates are engaged with each other for enabling movement of the endplates to a collapsed configuration, expanded configurations and tilted configurations. The endplates jointly define a graft material containment space when the endplates are in the collapsed configuration. The graft material containment space is at least partially encompassed by graft passage sidewalls extending from a perimeter edge portion of a graft window of at least one of the endplates. The endplate movement mechanisms each residing entirely in an interior space of the spinal implant external to the graft material containment space. Each of the endplate movement mechanisms is operably coupled to each of the endplates to enable adjustment of the endplates to the collapsed configuration, the expanded configurations and the tilted configurations.

In one or more embodiments, a spinal segment restoration device comprises first and second vertebrae support platforms and first and second movement mechanisms. The first and second vertebrae support platforms are engaged with each other for enabling movement of the vertebrae support platforms to a collapsed configuration, expanded configurations and tilted configurations. The vertebrae support platforms jointly define a graft passage when the vertebrae support platforms are in the collapsed configuration. The graft passage is at least partially bound by a plurality of graft passage sidewalls each extending from a perimeter edge portion of a graft window of a respective one of the vertebrae support platforms. The first and second movement mechanisms each residing entirely external to the graft passage. Each of the movement mechanisms is operably coupled to each of the vertebrae support platforms to enable adjustment of the vertebrae support platforms to the collapsed configuration, the expanded configurations and the tilted configurations.

In one or more embodiments, a translating linkage member of a first one of the endplate movement mechanisms is spaced away from a translating linkage member of a second one of the endplate movement mechanisms and the graft passage is positioned between the first and second ones of the endplate movement mechanisms.

In one or more embodiments, the translating linkage member of each of the endplate movement mechanisms includes first spaced-apart engagement members each translatably engaged with a respective elongated space within the first one of the endplates and second spaced-apart engagement members each translatably engaged with a respective elongated space within the second one of the endplates;

In one or more embodiments, the respective elongated space within the first one of the endplates extends generally parallel with a longitudinal centerline axis of the spinal implant device and the respective elongated space within the second one of the endplates extends in a skewed manner with respect to the longitudinal centerline axis of the spinal implant device.

In one or more embodiments, each of the endplate movement mechanisms reside entirely in a respective portion of an interior space of the spinal implant that is external to the graft material containment space.

In one or more embodiments, the graft material containment space is a graft passage.

In one or more embodiments, the interior space is at least partially encompassed by a plurality of graft passage sidewalls extending from a perimeter edge portion of a graft window of at least one respective one of the endplates.

In one or more embodiments, the interior space is at least partially encompassed by a plurality of graft passage sidewalls extending from a perimeter edge portion of a graft window of at least one respective one of the endplates.

In one or more embodiments, the graft passage sidewalls of at least one of the endplates extend contiguously around the graft passage.

In one or more embodiments, each of the endplates includes an anterior structural leg, a posterior structural leg and transverse structural legs coupled therebetween; the anterior structural leg and the posterior structural leg extend generally parallel to each other; the transverse structural legs are spaced part from each other; a first one of the endplate movement mechanisms extends at least partially along a length of a portion of the interior space partially defined by the anterior structural legs; and a second one of the endplate movement mechanisms extends along a length of a portion of the interior space partially defined by the posterior structural legs.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is a first perspective view showing a spinal implant device in accordance with an embodiment of the disclosures made herein in a collapsed configuration;

FIG. 2 is a second perspective view of the spinal implant device shown in FIG. 1;

FIG. 3 is a plan view of the spinal implant device shown in FIG. 1;

FIG. 4 is a first exploded view of the spinal implant device shown in FIG. 1;

FIG. 5 is a second exploded view of the spinal implant device shown in FIG. 1;

FIG. 6 is a third exploded view of the spinal implant device shown in FIG. 1;

FIG. 7 is a perspective view of the spinal implant device shown in FIG. 1 in an expanded configuration;

FIG. 8 is a perspective view of the spinal implant device shown in FIG. 1 in a tilted configuration;

FIG. 9 is a cross-sectional view taken along the line 9-9 in FIG. 1;

FIG. 10 is a cross-sectional view taken along the line 10-10 in FIG. 1; and

FIG. 11 is a cross-sectional view taken at line 11-11 in FIG. 8.

### DETAILED DESCRIPTION

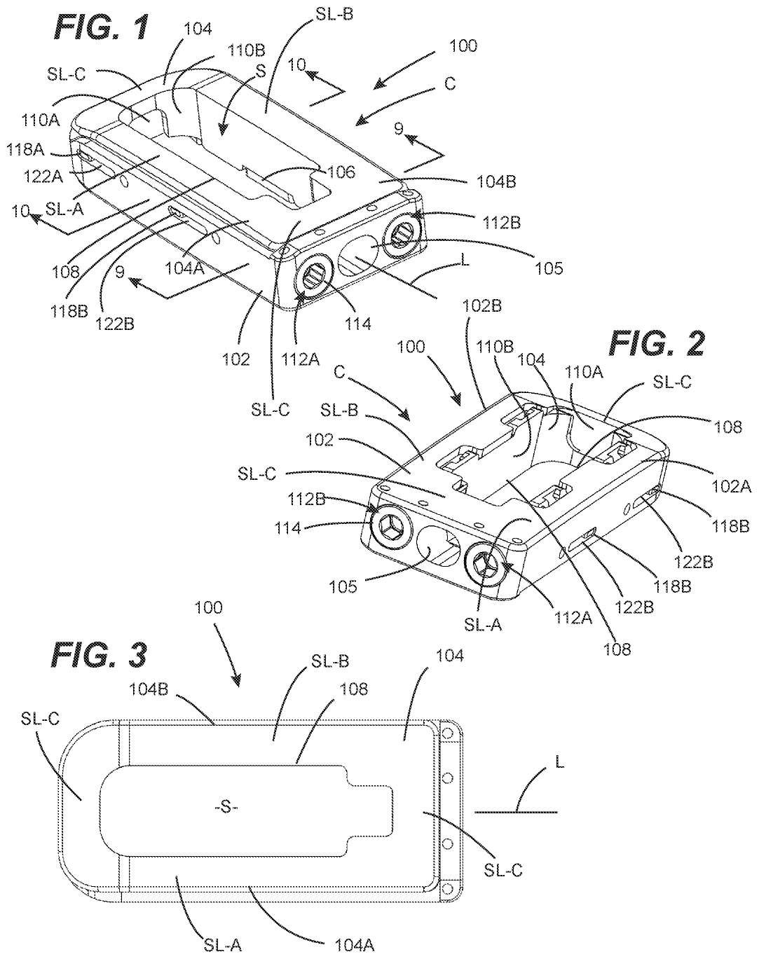

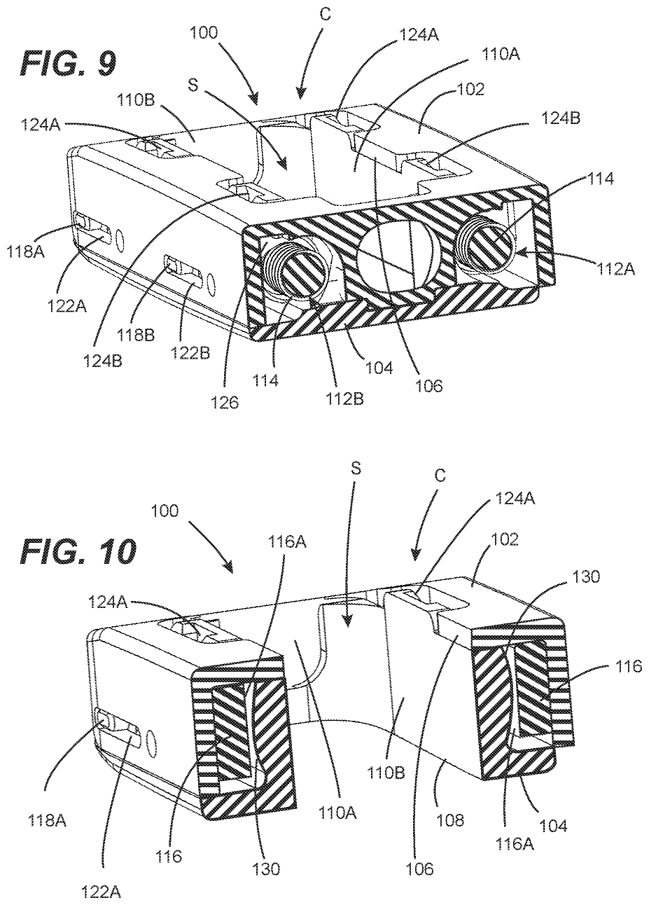

Referring now to the FIGS. 1-10, an implant configured in accordance with one or more embodiments of the disclosures made here (i.e., implant device 100) is shown. In one or more preferred embodiments, the implant device 100 is a spinal implant useful for correcting ailments associated with deterioration of an intervertebral disc and/or misalignment of an intervertebral disc and its corresponding adjacent vertebral bones. Advantageously, the implant device 100 includes an unobstructed graft material containment space S. In this manner, the implant device 100 has a structural configuration in which an overall plan view area of the graft material containment space S is devoid of elements thereof that enable movement and adjustment of the expandable interbody devices between a collapsed configuration C and displaced configurations. Thus, when the implant device 100 is in (and sufficiently near) the collapsed configuration C, the graft material containment space S is free of obstructions that would otherwise limit the available volume of graft material and associated bony material growth that may form within the graft material containment space S.

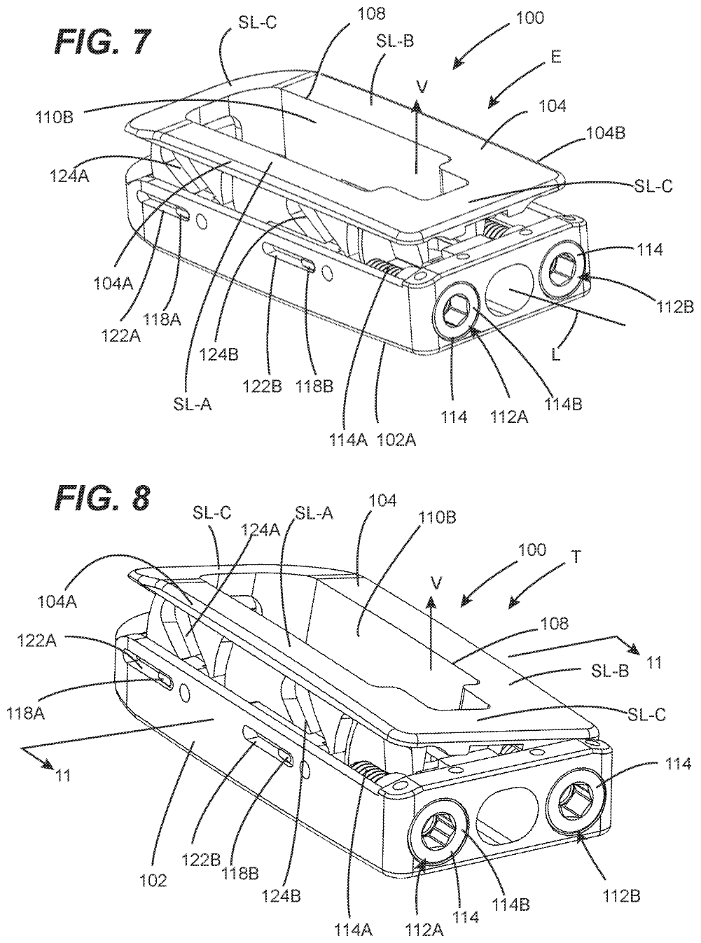

The implant device 100 includes a first endplate 102 and a second endplate 104 that each serve as a respective vertebrae support platform. The endplates 102, 104 are engaged with each other for enabling movement of the endplates 102, 104 to a collapsed configuration C (FIGS. 1-3), an expanded configuration E (FIG. 7) and a tilted configuration T (FIG. 8). In the collapsed configuration C, the endplates 102, 104 are in a seated position with respect to each other. In the expanded configuration E, as shown in FIG. 7, an entire portion of the first and second endplates 102, 104 are in a position that is uniformly displaced from each other (i.e., a vertically displaced from each other). In the tilted configuration T, as shown in FIG. 8, a first edge portion 102A,104A of the first and second endplates 102, 104 and a second edge portion 102B,104B of the first and second endplates 102, 104 are in an expanded configuration different than each other. For example, the first edge portion 102A,104A (e.g., posterior) of the first and second endplates 102, 104 can be edge portions that are fully or partially displaced (i.e., expanded) from each other, whereas the second edge portion 102B,104B (e.g., anterior) of the first and second endplates 102, 104 can be non-displaced from each other (i.e., fully collapsed) or displaced from each other by a lesser amount than the first edge portion 102A,104A of the first and second endplates 102, 104. Alternatively, unequal displacement of the first edge portion 102A,104A (e.g., posterior) of the first and second endplates 102, 104 and the second edge portion 102B,104B (e.g., anterior) of the first and second endplates 102, 104 can be reversed. The aforementioned tilted configuration C is commonly referred to as a lordotic configuration (or kyphotic in some cases), which is utilized for providing lordosis/kyphosis adjustment of adjacent vertebra of a spine.

As best shown in FIGS. 1-3, the endplates 102, 104 jointly define a graft material containment space S when the endplates 102, 104 are in the collapsed configuration C. Each of the endplates 102, 104 has a respective graft window 106, 108 extending therethrough for enabling access to the graft material containment space S. In this respect, the graft material containment space S is a graft passage extending through the implant device 100. The graft material containment space S is at partially or fully encompassed by graft passage sidewalls 110A, 110B extending from a perimeter edge portion of a respective one of the graft windows 106, 108.

In one or more embodiments, the graft passage sidewalls 110A, 110B of at least one of the endplates 102, 104 extend contiguously around the graft material containment space S. For example, as shown, the graft passage sidewalls 110B of the second endplate 104 extend contiguously around the graft material containment space S as well as the graft passage sidewalls 110A, 110B of the first and second endplates 102, 104 jointly extending contiguously around the graft material containment space S.

As best shown in FIGS. 1-3, each of the endplates 102, 104 includes a first structural leg SL-A adjacent the respective first edge portion 102A,104A (e.g., posterior) of the respective one of the endplates 102, 104, a second structural leg SL-B adjacent the respective second edge portion 102B,104B (e.g., anterior) of the respective one of the endplates 102, 104 and transverse structural legs SL-C coupled therebetween. The first structural leg SL-A and the second structural leg SL-B of each of the endplates 102, 104 extend generally parallel to each other. The transverse structural legs SL-C are spaced part from each other. When the implant device is in the collapsed configuration C, a first one of the endplate movement mechanisms 112A, 112B extends at least partially along a length of a portion of an interior space of the implant device 100 (i.e., space encompassed by exterior surfaces of the implant device 100) partially defined by the first structural legs SL-A and a second one of the endplate movement mechanisms 112A, 112B extends along a length of a portion of the interior space partially defined by the second structural legs SL-B

The implant device 100 includes a first endplate movement mechanism 112A and a second endplate movement mechanism 112B. The first and second endplate movement mechanisms 112A, 112B are laterally spaced-apart from each other. The graft material containment space S is positioned between the first and second endplate movement mechanisms 112A, 112B. As discussed below in greater detail and as is clearly shown in the drawing figures, each of the endplate movement mechanisms 112A, 112B advantageously residing entirely in an interior space of the implant device 100 that is external to the graft material containment space S. In this manner, when the implant device 100 is in (and sufficiently near) the collapsed configuration C, no part of the endplate movement mechanisms 112A, 112B reside within the graft material containment space S and the graft material containment space S is free of any other obstruction that would otherwise limit the available volume of graft material and associated bony material growth that may form within the graft material containment space S.

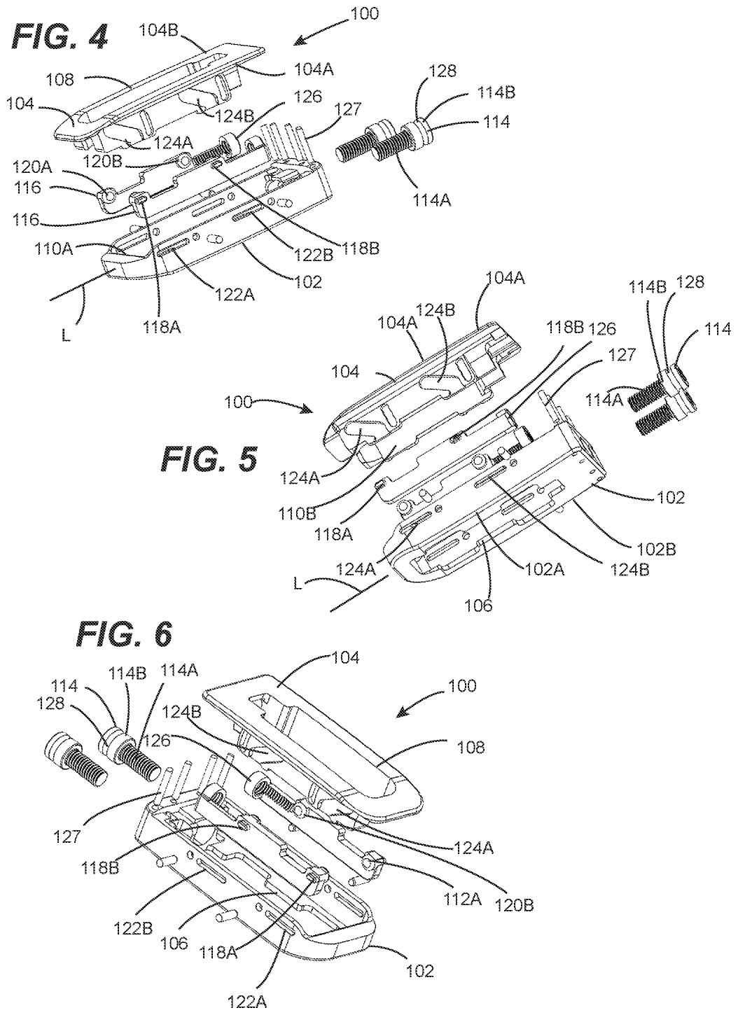

As best shown in FIGS. 4-6, each of the endplate movement mechanisms 112A, 112B includes an adjuster 114 and a translating linkage member 116. Each translating linkage member 116 includes first spaced-apart engagement members 118A, 118B and second spaced-apart engagement members 120A, 120B. The first engagement members 118A, 118B are spaced-apart from each other and are each translatably engaged with a respective elongated slot 122A, 122B (i.e., elongated spaces) within the first endplate 102. The second engagement members 120A, 120B are spaced-apart from each other and are each translatably engaged with a respective elongated slot 124A, 124B (i.e., elongated spaces) within the second endplate 104. The elongated slots 122A, 122B within the first endplate 102 extends generally parallel with a longitudinal centerline axis L of the implant device 100 and the elongated slot 124A, 124B within the second endplate 104 extends in a skewed manner with respect to the longitudinal centerline axis L and parallel with respect to each other.

The first engagement members 118A, 118B and the elongated slot 122A, 122B of the first endplate 102 jointly define an interface structure adapted for constraining displacement of each of the translating linkage member 116 to be substantially parallel with the longitudinal centerline axis L of the implant device 100. The second engagement members 120A, 120B and the elongated slot 124A, 124B of the second endplate 104 jointly define an interface structure adapted for constraining displacement of the respective portion of the second endplate 104 to be vertical with respect to the first endplate 102 in response to axial translation of the respective one of the translating linkage members 116. In view of the disclosures made herein, a skilled person will understand that the structural association of the engagement members 118A, 118B and the elongated slot 122A, 122B of the first endplate 102 may be reversed (i.e., each integral with the opposite structural element) and that the structural association of the engagement members 120A, 120B and the elongated slot 124A, 124B of the second endplate 104 may be reversed (i.e., each integral with the opposite structural element). Furthermore, in view of the disclosures made herein, a skilled person will appreciate other structural arrangements to be used in place of the endplate movement mechanisms 112A, 112B and mating structures of the endplates 102, 104.

The adjuster 114 of each of the endplate movement mechanisms 112A, 112B includes a shank portion 114A and a head portion 114 B fixedly attached to the shank portion 114A. In one or more or more embodiments, the adjuster 114 is a threaded fastener such as, for example, a screw. The shank portion 114A of an adjuster 114 is interlockedly engaged (e.g., threadedly) with receptacle portion 126 of a respective one of the translating linkage members 116. The head portion 114B is affixed to the first endplate 102 by spaced-apart retention members 127 (e.g., pins) that are coupled to the first endplate 102 and that engage a mating feature 128 (e.g., groove) of the head portion 114B for enabling rotation of the adjuster 114 relative to the first endplate 102 but inhibiting its unrestricted axial displacement thereto.

Referring to FIGS. 1, 2, 7 and 8, in operation, rotation of the shank portion 114A results in a corresponding (e.g., relational) axial translation of the translating linkage members 116 with respect to each of the endplates 102, 104. Rotation of the shank portion 114A is implemented through rotation of the head portion 114B such as via engagement of an interlock portion of an adjustment tool with a mating portion of the head portion 114B and rotation of at least the interlock portion of the adjustment tool. The axial translation of the translating linkage members 116 is constrained to be along the longitudinal centerline axis L by virtue of the first engagement members 118A, 118B each being translatably engaged with the respective elongated slot 122A, 122B within the first endplate 102. The axially constrained translation of either one of the adjustors 114 in vertical displacement V of an associated portion of the second endplate 104 with respect to the first endplate 102 by virtue of the second engagement members 120A, 120B are each translatably engaged with the respective elongated slot 124A, 124B and each of the elongated slot 124A, 124B being skewed with respect to the longitudinal centerline axis L.

For example, rotation of the adjustor 114 adjacent to the first edge portion 102A, 104A of the first and second endplates 102, 104 causes a corresponding vertical displacement V of the first edge portion 104A of the second endplate 104 with respect to the first edge portion 102A of the first endplate 102 and rotation of the adjustor 114 adjacent to the second edge portion 102B, 104B of the first and second endplates 102, 104 causes a corresponding vertical displacement V of the second edge portion 104B of the second endplate 104 with respect to the second edge portion 102B of the first endplate 102. Thus, the first and second edge portions 104A, 104B of the second endplate 104 may be independently adjusted with respect to the respective adjacent one of the edge portions 102A, 102B of the first endplate 102. Direction of rotation of the adjustor 114 dictates direction of the vertical displacement. Through such adjustment of the adjustors 114, the first and second endplates 102, 104 can be selectively mobilized to the collapsed configuration, expanded configuration and tilted configuration. Selective adjustment of one or both of the endplate movement mechanisms 112A, 112B enables selective adjustment of the endplates 102, 104 from the collapsed configuration C to a displaced configuration (i.e., expanded and/or tilted) for providing restoration of adjacent bony structures (e.g., vertebrae). In the tilted configuration, the first and second edge portions 104A, 104B of the second endplate 104 may be independently adjusted with respect to the respective adjacent one of the edge portions 102A, 102B of the first endplate 102.

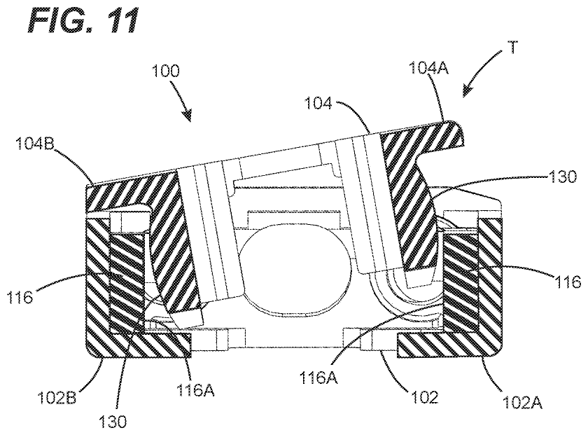

Referring to FIGS. 8 and 11, direction of tilt is controlled by rotation of the adjustor 114 of the first endplate movement mechanism 112A while maintaining the adjustor 114 of the second endplate movement mechanism 112B in a static position. Through such adjustment of the adjustors 114, the first and second endplates 102, 104 can be selectively placed in a desired tilted configuration (e.g., the tilted configuration T). As shown in FIG. 11, during tilting adjustment (i.e., angulation) via adjustment of the adjustor 114 of the first endplate movement mechanism 112A, a flat surface 116A of the translating linkage 116 of the second endplate movement mechanism 112B is tangential to an adjacent arcuate surface 130 of the second endplate 104 (i.e., the flat surface 116A of the translating linkage member 116 engages the adjacent arcuate surface 130 of the second endplate 104). Advantageously, compared to a device having a flat surface in place of the arcuate surface 130, the arcuate surface 130 provides contact surface guided angulation and clearance to the adjacent translating linkage 116 at the terminal end of tilting adjustment. The increased clearance beneficially results in an increased range of tilting adjustment by virtue of a portion of the arcuate contact surface existing in place of a flat surface that bind with translating linkage member 116 at the terminal end of tilting adjustment.

In view of the disclosures made herein, a skilled person will appreciate the advantageous and beneficial aspect of the structure enabling movement of the endplates. Specifically, endplate movement mechanisms in accordance with one or more embodiments of the disclosure made herein enabling endplate expansion and retraction functionality in a manner whereby retraction does not require external compressive loading being exerted on the first and second endplates. For example, operable coupling of the endplates 102 and 104 and endplate movement mechanisms 112A, 112B of the implant device 100 are implemented in a “pinned manner ” by virtue of the engagement members 118A, 118B, of each endplate movement mechanism 112A, 112B each being captured within a mating one of the elongated slots 122A, 122B of the first endplate 102 and the engagement members 120A, 120B of each endplate movement mechanism 112A, 112B each being captured within a mating one of the elongated slot 124A, 124B of the second endplate 104. Accordingly, actuation of either of the endplate movement mechanisms 112A, 112B for expansion or retraction causes a corresponding relative movement of the second endplate 104 relative to the first endplate 102 irrespective of external compressive loading of the endplates 102, 104 via opposing engaged vertebrae engaged therewith. Such operability is advantageous as it cannot be presumed that the endplates 102, 104 will be under external compressive loading via opposing engaged vertebrae.

As shown in FIGS. 1 and 2, the first endplate 102 of the implant device 100 includes a passageway 105 located between the endplate movement mechanism 112A, 112B. The passageway 105 extends between the external surface of the first endplate 102 and may be used as a passageway for deposit of grafting material associated with facilitating growth of bony tissue therein. The passageway may be configured as a threaded aperture for receiving a mating threaded portion of a fastener (screw, stud, etc.) engaged therein. It is well known that such a threaded passageway of an implant body may be used for enabling engagement of an implant inserter with the implant device. Advantageously, particularly when the implant device 100 exhibits a relatively large amount of lordotic adjustment (angulation), such a threaded passageway may be used for engagement of the fastener securing a securement body (e.g., plate or other implant securing structure) to the implant device 100 to prevent unwanted (e.g., unrestricted) movement of the implant device relative to adjacent vertebrae (i.e., bone tissue).

In one or more embodiments, an implant device in accordance with the disclosures made herein may be a lumbar spinal interbody fusion device designed to be used in the lumbar 2/3, lumbar 3/4, lumbar 4/5, and Lumbar 5/Sacrum 1 interbody spaces. Such a device facilitates fusion of the interbody space at the aforementioned levels. This device facilitates placement into the aforementioned lumbar interbody spaces with less traumatic vertebral body impulse forces than currently available interbody fusion devices. This device is inserted into the lumbar interbody space in a collapsed configuration (e.g., fully or partially collapsed) and is capable of in-vivo expansion after such placement into the interbody space. Contact surfaces of this device are capable of expanding in a parallel or a variable angle fashion to facilitate restoration of spinal segmental anatomic alignment. The endplate angle of this device can be adjusted in-vivo ranging from zero degrees parallel to a kyphotic or lordotic final angle to customize an optimal fit in individualized patient anatomy.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the invention in all its aspects. Although the invention has been described with reference to particular means, materials and embodiments, the invention is not intended to be limited to the particulars disclosed; rather, the invention extends to all functionally equivalent technologies, structures, methods and uses such as are within the scope of the appended claims.

What is claimed is:

1. An implant device, comprising:
spaced-apart endplates engaged with each other for enabling movement of the endplates to a collapsed configuration and to displaced configurations, wherein the endplates jointly define a graft passage when the endplates are in the collapsed configuration and wherein the graft passage is at least partially encompassed by a plurality of graft passage sidewalls each extending from a perimeter edge portion of a graft window of a respective one of the endplates; and
endplate movement mechanisms each located entirely external to the graft passage and operably coupled to each of the endplates to enable adjustment of the endplates to the collapsed configuration and to the displaced configurations.

2. The implant device of claim 1 wherein:
a translating linkage member of a first one of the endplate movement mechanisms is spaced away from a translating linkage member of a second one of the endplate movement mechanisms; and
the graft passage is positioned between the first and second ones of the endplate movement mechanisms.

3. The implant device of claim 2 wherein:
the translating linkage member of each of the endplate movement mechanisms includes first spaced-apart engagement members each translatably engaged with a respective elongated space within the first one of the endplates and second spaced-apart engagement members each translatably engaged with a respective elongated space within the second one of the endplates;
the respective elongated space within the first one of the endplates extends generally parallel with a longitudinal centerline axis of the spinal implant device; and
the respective elongated space within the second one of the endplates extends in a skewed manner with respect to the longitudinal centerline axis of the spinal implant device.

4. The implant device of claim 3 wherein each of the endplate movement mechanisms reside entirely in a respective portion of an interior space of the spinal implant that is external to the graft passage.

5. The implant device of claim 4 wherein the interior space is at least partially encompassed by a plurality of graft passage sidewalls extending from a perimeter edge portion of a graft window of at least one respective one of the endplates.

6. The implant device of claim 1 wherein each of the endplate movement mechanisms reside entirely in a respective portion of an interior space of the spinal implant that is external to the graft passage.

7. The implant device of claim 6 wherein the interior space is at least partially encompassed by a plurality of graft passage sidewalls extending from a perimeter edge portion of a graft window of at least one respective one of the endplates.

8. The implant device of claim 7 wherein the graft passage sidewalls of at least one of the endplates extend contiguously around the graft passage.

9. The implant device of claim 1 wherein the graft passage sidewalls of at least one of the endplates extend contiguously around the graft passage.

10. The implant device of claim 1 wherein:
each of the endplates includes an anterior structural leg, a posterior structural leg and transverse structural legs coupled therebetween;
the anterior structural leg and the posterior structural leg extend generally parallel to each other;
the transverse structural legs are spaced part from each other;
a first one of the endplate movement mechanisms extends at least partially along a length of a portion of the interior space partially defined by the anterior structural legs; and
a second one of the endplate movement mechanisms extends along a length of a portion of the interior space partially defined by the posterior structural legs.

11. The implant device of claim 10 wherein the graft passage sidewalls of at least one of the endplates extend contiguously around the graft passage.

12. The implant device of claim 1 wherein:
a translating linkage member of a first one of the endplate movement mechanisms is spaced away from a translating linkage member of a second one of the endplate movement mechanisms; and each of the translating linkage members has a flat side surface thereof engaged with an arcuate side surface of an adjacent one of the endplates.

13. An implant device, comprising:
first and second endplates engaged with each other for enabling movement of the endplates to a collapsed configuration, expanded configurations and tilted configurations, wherein the endplates jointly define a graft material containment space when the endplates are in the collapsed configuration and wherein the graft material containment space is at least partially encompassed by graft passage sidewalls extending from a perimeter edge portion of a graft window of at least one of the endplates; and
spaced-apart endplate movement mechanisms each residing entirely in an interior space of the spinal implant external to the graft material containment space, wherein each of the spaced-apart endplate movement mechanisms is operably coupled to each of the endplates to enable adjustment of the endplates to the collapsed configuration, the expanded configurations and the tilted configurations.

14. The implant device of claim 13 wherein:
a translating linkage member of a first one of the spaced-apart endplate movement mechanisms is spaced away from a translating linkage member of a second one of the spaced-apart endplate movement mechanisms; and
the graft material containment space is located between the first and second ones of the spaced-apart endplate movement mechanisms.

15. The implant device of claim 14 wherein:
the translating linkage member of each of the spaced-apart endplate movement mechanisms includes first spaced-apart engagement members each translatably engaged with a respective elongated space within the first one of the endplates and second spaced-apart engagement members each translatably engaged with a respective elongated space within the second one of the endplates;
the respective elongated space within the first one of the endplates extends generally parallel with a longitudinal centerline axis of the spinal implant device; and
the respective elongated space within the second one of the endplates extends in a skewed manner with respect to the longitudinal centerline axis of the spinal implant device.

16. The implant device of claim 13 wherein:
each of the spaced-apart endplate movement mechanisms reside entirely in a respective portion of an interior space of the spinal implant that is external to the graft passage; and
the interior space is at least partially encompassed by a plurality of graft passage sidewalls extending from a perimeter edge portion of a graft window of at least one respective one of the endplates.

17. The implant device of claim 13 wherein:
each of the endplates includes an anterior structural leg, a posterior structural leg and transverse structural legs coupled therebetween;
the anterior structural leg and the posterior structural leg extend generally parallel to each other;
the transverse structural legs are spaced part from each other;
a first one of the spaced-apart endplate movement mechanisms extends at least partially along a length of a portion of the interior space partially defined by the anterior structural legs; and
a second one of the spaced-apart endplate movement mechanisms extends along a length of a portion of the interior space partially defined by the posterior structural legs.

18. A spinal segment restoration device, comprising:
first and second vertebrae support platforms engaged with each other for enabling movement of the vertebrae support platforms to a collapsed configuration, expanded configurations and tilted configurations, wherein the vertebrae support platforms jointly define a graft passage when the vertebrae support platforms are in the collapsed configuration and wherein the graft passage is at least partially bound by a plurality of graft passage sidewalls each extending from a perimeter edge portion of a graft window of a respective one of the vertebrae support platforms; and
first and second movement mechanisms each residing entirely external to the graft passage, wherein each of the movement mechanisms is operably coupled to each of the vertebrae support platforms to enable adjustment of the vertebrae support platforms to the collapsed configuration, the expanded configurations and the tilted configurations.

19. The spinal segment restoration device of claim 18 wherein:
a translating linkage member of a first one of the movement mechanisms is spaced away from a translating linkage member of a second one of the movement mechanisms; and
the graft passage is positioned between the first and second ones of the movement mechanisms.

20. The spinal segment restoration device of claim 18 wherein each of the endplate movement mechanisms reside entirely in a respective portion of an interior space of the spinal segment restoration device that is external to the graft passage.

21. The spinal segment restoration device of claim 18 wherein:
each of the vertebrae support platforms includes an anterior structural leg, a posterior structural leg and transverse structural legs coupled therebetween;
the anterior structural leg and the posterior structural leg extend generally parallel to each other;
the transverse structural legs are spaced part from each other;
a first one of the movement mechanisms extends at least partially along a length of a portion of the interior space partially defined by the anterior structural legs; and
a second one of the movement mechanisms extends along a length of a portion of the interior space partially defined by the posterior structural legs.
